(19) United States
(12) Patent Application Publication
(10) Pub. No.: US 2007/0185506 A1
(43) Pub. Date: Aug. 9, 2007

(54) Medical instruments and methods for using the same

(76) Inventor: Kelly Jackson, Draper, UT (US)

Correspondence Address: Kirton & McConkie, P.O. Box 45120, Salt Lake City, UT, 84145-0120, US

(21) Appl. No.: 10/911,977

(22) Filed: Aug. 4, 2004

Related U.S. Application Data
(60) Provisional application No. 60/492,375, filed on Aug. 4, 2003.

(51) Int. Cl. A61B 17/08

(52) U.S. Cl. 606/151

(57) ABSTRACT

A medical instrument used to position a surgical mesh sheet to a surface of the human body. The instrument contains a mechanism for holding the mesh sheet against the tissue surface in more than two locations during the attaching process, making attachment of the sheet to the surface quicker and easier. The instrument also contains a mechanism for grasping and then quickly and easily releasing the mesh sheet from the instrument once it is in place. The invention can also be used to hold the mesh in position without the use of the mechanism that grips the mesh.

### CROSS-REFERENCE TO RELATED APPLICATION

This application claims priority of U.S. Patent Application Ser. No. 60/492,375, filed on Aug. 4, 2003, the disclosure of which is incorporated herein by reference.

### FIELD OF THE INVENTION

The invention generally relates to medical devices and methods for using such medical devices. Even more particularly, the invention relates to medical instruments used in laparoscopic hernia repair and methods for reinforcing and/or repairing damaged tissue or muscle walls that accompany hernias or similar medical conditions.

### BACKGROUND OF THE INVENTION

Hernias are common injuries that are caused by people straining their abdominal muscles. A hernia is a defect (or rip or tear) in the abdominal wall through which a portion of the intra-abdominal contents protrude, thereby causing discomfort and an unsightly, visible bulge in the abdomen.

When such a hernia defect occurs, corrective surgery has typically required opening the abdominal cavity by surgical incision through the major abdominal muscles.

While this technique effectively corrects the hernia defect, it has the disadvantages of requiring a lengthy hospital stay and an extended period of recuperation. Accordingly, alternative techniques that are less traumatic to the patient and provide for more rapid recovery have been sought.

Laparoscopy is one such alternative technique. In laparoscopy, surgical tools are inserted through tiny openings in the abdominal wall called ports. Through one port is inserted the viewing device. The various surgical instruments are inserted through other ports to perform the surgery that otherwise would be performed through an open incision in the abdominal wall. Because the laparoscopic surgical techniques require only very small holes known as ports, the laparoscopic instruments must be of a size and configuration to be inserted though these ports. These ports commonly have an inside diameter of 5 mm or 10 mm.

Some efforts have been made to incorporate laparoscopic techniques into hernia repair surgery. See, for example, U.S. Pat. No. 6,425,924, the disclosure of which is incorporated herein by reference. These efforts have generally retracted the intra-abdominal contents away from the hernia defect and then inserted a bundle of surgical mesh or other material into the defect. In one technique, a patch of surgical mesh is placed over the defect and then is clipped or stapled into place. This technique has been utilized with some success, but has suffered from not being able to spread a patch of surgical mesh and hold it in place smoothly and relatively rigidly during the fastening procedure. Lacking any specific device for the purpose, it is common for the surgeon to position the mesh with a standard laparoscopic grasper, which holds the mesh at only one point.

One problem with the surgical mesh sheet is that the current instruments have a difficulty placing and holding the mesh sheet against the flat surface while the surgeon is attaching it. Generally, such medical instruments, taught for holding mesh, are only able to hold the mesh sheet in a single or a pair of locations by hooks, thus only facilitating its attachment by using one or two specific locations. See, for example, U.S. Pat. No. 5,383,477, the disclosure of which is incorporated herein by reference. This procedure proves to be very difficult because the mesh tends to not stay flat against the flat surface, i.e., the sheet can “flap” around since the medical instrument is only holding it at one or two locations. As well, the hooks of the medical instrument used to “hold” the mesh sheet do not easily release the mesh sheet when desired. Further, such medical instruments are not easily used with mesh sheets that have a backing or coating (such as silicone or Teflon) because they don't effectively grip the mesh sheet through the layer.

### SUMMARY OF THE INVENTION

The invention provides a medical instrument used to position a surgical mesh sheet to a surface of the human body. The instrument contains a mechanism for holding the mesh sheet against the tissue surface in more than two locations during the attaching process, making attachment of the sheet to the surface quicker and easier. The instrument also contains a mechanism for grasping and then quickly and easily releasing the mesh sheet from the instrument once it is in place. The invention can also be used to hold the mesh in position without the use of the mechanism that grips the mesh.

### BRIEF DESCRIPTION OF THE DRAWINGS

The following description of the invention can be understood in light of FIGS. 1-5 which depict various aspects of the medical instruments of the invention.

FIG. 1 illustrates the medical instrument in one aspect of the invention;

FIG. 2 depicts the medical instrument in another aspect of the invention;

FIG. 3 illustrates an example of a mesh sheet that can be used in combination with the medical instrument in one aspect of the invention;

FIG. 4 depicts a close-up view of one part of the medical instrument in one aspect of the invention; and

FIG. 5 shows a close-up view of the mesh sheet in combination with another part of the medical instrument in one aspect of the invention.

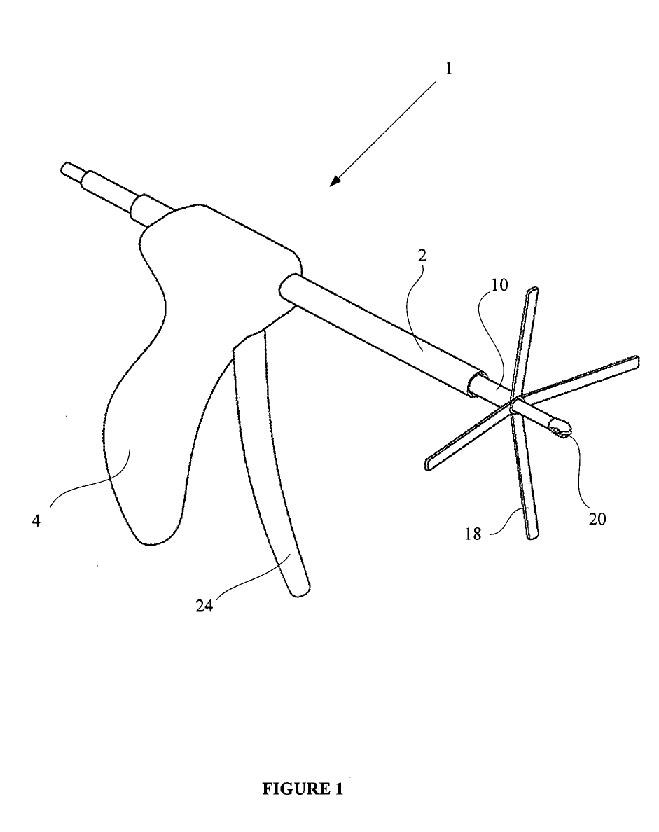

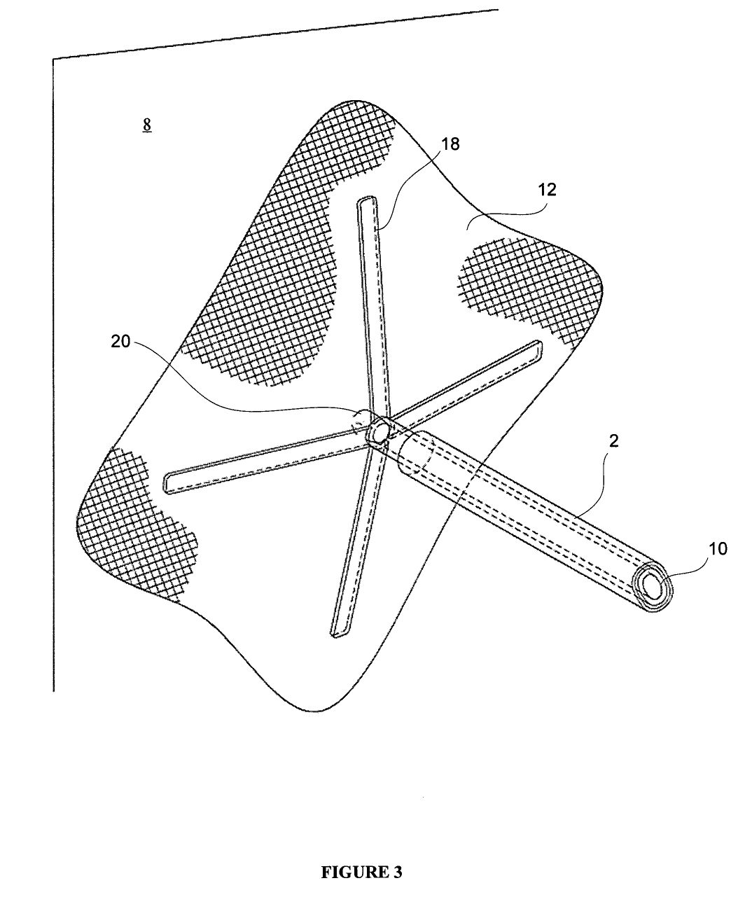

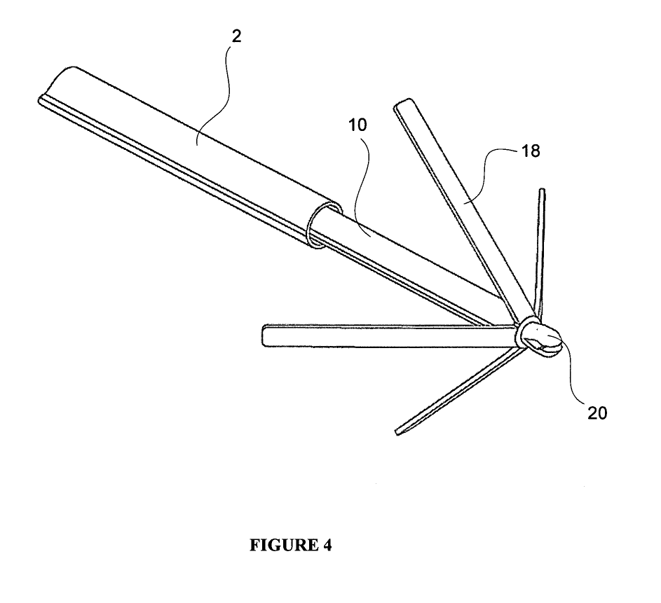

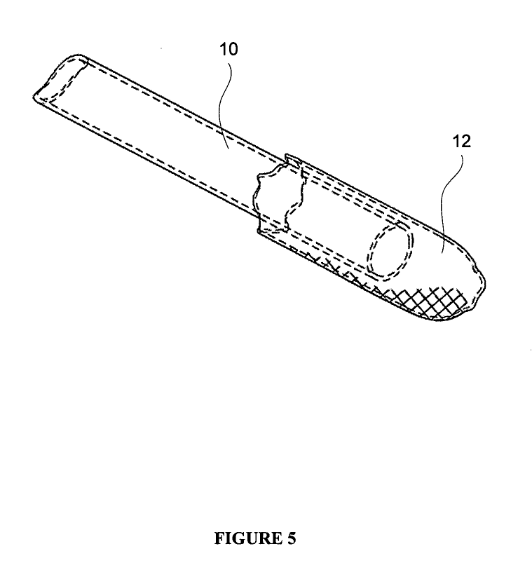

FIGS. 1-5 illustrate specific aspects of the invention, and are a part of the specification. Together with the following description, the Figures demonstrate and explain the principles of the invention. The Figures presented in conjunction with this description are views of only particular-rather than complete-portions of the invention. In the Figures, the physical dimensions may be exaggerated for clarity. The same reference numerals in different drawings represent the same element, and thus their descriptions will be omitted.

### DETAILED DESCRIPTION OF THE INVENTION

The following description provides specific details in order to provide a thorough understanding of the invention. The skilled artisan, however, would understand that the invention can be practiced without employing these specific details. Indeed, the invention can be practiced by modifying the illustrated method and resulting product and can be used in conjunction with apparatus and techniques conventionally used in the industry.

For example, the invention is described for use with hernia operations, but can be used for any surgery/operation where a pliable or flexible piece of material needs to be held in place while it is attached to a surface, such as vascular repair or in cases of urinary incontinence. As described above, the invention includes a medical instrument capable of grasping and positioning a pliable or flexible piece of material against a part of the body while the material is attached to that part of the body quickly and easily. The instrument of the invention can be used with any internal surface of the body can be used, especially a substantially flat inter surface.

The pliable piece of material can be any flexible material known in the art that can be placed and used within an internal portion of the human body, such as an internal surface. In one aspect of the invention, any known sheet 12 of a mesh material used for such purposes can be employed in the invention. Mesh materials are typically made from sterile woven polyester materials. Such materials are also thin, soft, pliable or flexible, allowing them to easily conform to the part of the body to which they will be attached (i.e., abdominal wall). Mesh materials are also quite strong to help repair the hernia completely and immediately. Examples of such mesh sheets include ProLite, Marlex, and Prolene.

The medical instrument of the invention positions such a pliable piece of material against a part of the body while the material is attached to that part of the body quickly and easily. Any instrument operating in such a manner can be used in the invention, such as the instrument depicted in the Figures and described below. In this aspect of the invention, the medical instrument 1 contains holding means, handling means, steering means, triggering means, optional grasping means, optional gripping means, and optional attaching means, as described in more detail below.

This instrument 1 can be fabricated of any substantially rigid material. Examples of such rigid materials include hard plastics such as polycarbonate, polypropylene, acrylic, stainless steel, or combinations thereof. The instrument 1 contains an outer housing 2 with an open end. The outer housing 2 is configured so that it can be inserted and removed through any surgical port or trochar, or for insertion into a bodily cavity. For example, the outer housing 2 could be configured to fit through a 10 mm trochar. While the outer housing 2 can have any shape such as elliptical, square, polygonal, or the like, it is typically configured with a substantially cylindrical shape.

The instrument 1 also contains an inner housing 10 that extends generally within housing 2 and has an open end contained with the end of the outer housing. The inner housing 10 can be moved within the outer housing 2 by sliding the elements related to one another. Accordingly, the inner housing is configured with a size and a shape that allows such movement within the inner housing 10.

The inner housing 10 contains the holding means, that portion of the instrument that is used to releasably hold the flexible material (i.e., mesh sheet) against the portion of the body (i.e., the abdominal wall or other body surface 8) in a plurality of locations. Any holding means that can accomplish this function can be used in the invention. The holding means is typically stored in a compressed configuration in the instrument until it is situated near the internal body surface, i.e., near open end of the instrument 1.

The holding means contains a number of contact mechanisms. The contact mechanism is that part of the holding means which actually contacts the mesh sheet. The contact mechanism must be capable of contacting the mesh sheet until it is attached to the abdominal wall. The contact mechanism must then be able to be easily removed from the mesh sheet and return to the medical instrument. During that process, the remainder of the holding means remains in the inner housing 10.

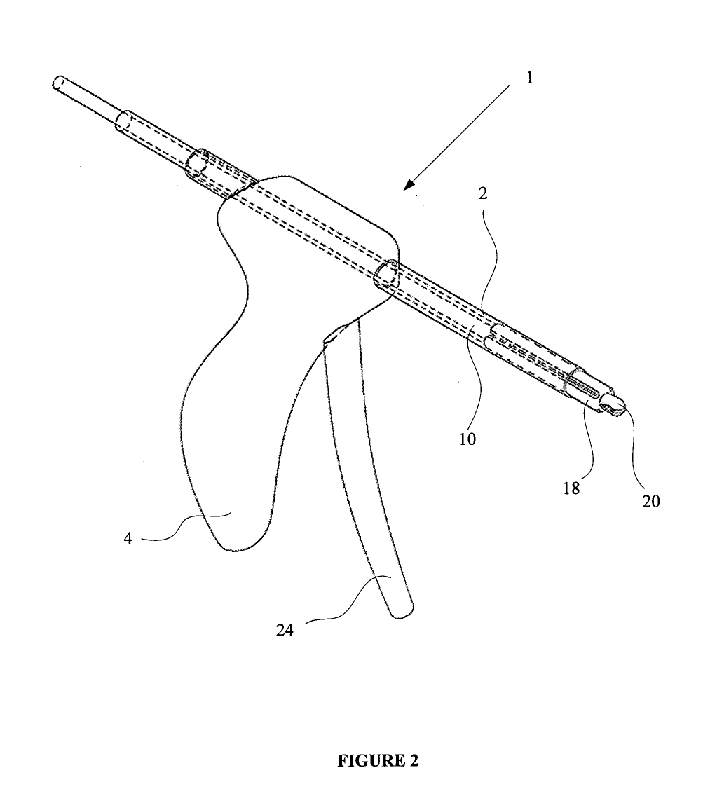

In one aspect of the invention, the contact mechanism is stored in a compressed form in the instrument 1 and expands when needed by the user. Using this configuration allows it to be inserted through very small laparoscopic ports. As shown in FIG. 2, the contact mechanism can be stored in the inner housing 10 (usually near the open end) of the instrument 1. When triggered, the contact mechanism then expands out of the open end of the inner housing 10, along with the mesh sheet. The contact mechanism expands in a manner that also expands the mesh sheet 12. The contact mechanism then holds the mesh sheet in place in the desired location. After the mesh sheet is attached, the contact mechanism can be retracted into the inner housing 10, thereby allowing removal of the instrument 1 from the port.

The contact mechanism can be any apparatus that operates in the manner described above. In one aspect of the invention, the contact mechanism comprises a plurality of wires. In another aspect of the invention, the contract mechanism comprises an umbrella-like structure with any shape (e.g., circular, square, polygonal, etc.). In yet another aspect of the invention, the contact mechanism is a plurality of ribs and/or corrugations. Any of these contact mechanisms, including a combination of these mechanisms, can be used in invention. The type of elements 18 (ribs, wires, corrugations, umbrella structure, or other extending parts) of the contact mechanism are selected for the quickest attachment of the mesh sheet to the body surface.

The number of elements 18 is also selected with the same goal in mind. While this number is theoretically unlimited, it generally can range from 3 to 12. Typically, this number can range from 4 to 6. The width of the elements is also elected with this goal in mind, but generally ranges from about 2.5 mm to about 10 mm.

The contact mechanism can be shortened or lengthened as needed. In one aspect of the invention, the length of the contact mechanism depends on the expected distance between the instrument 1 and the desired portion of the body. In this aspect of the invention, the length of the contact mechanism can range from about 10 mm to about 60 mm. In one aspect of the invention (such as where the contact mechanism is longer than needed), the whole length of the contact mechanism need not be extended beyond the inner housing, thereby leaving a portion of the contact mechanism in the inner housing.

In another aspect of the invention, not all parts or other elements of the contact mechanism need be the same length. Indeed, certain parts (certain wires) of the contact mechanism can be short while other parts (other wires) can be long. As well, a first number of parts could be a first length, a second number could be a second length, a third number could be a third length, etc. This aspect is especially useful since, in certain instances, it could maximize contact between the mesh sheet and the contact mechanism, thereby maximizing the contact between the mesh sheet and the body surface.

The contact mechanism holds the mesh sheet in place until it is attached to the abdominal wall or other body surface. At that point, the contact mechanism can be retracted into the inner housing 10 and the instrument is then withdrawn from the port. In one aspect of the invention, the instrument can grasp the mesh in generally the center of the piece of mesh using grasper means. The instrument then releases the mesh at this generally point while the elements 18 of the contact mechanism continue to hold the mesh sheet in position against the surface.

The grasper means used in the invention can be any means known in the art, including the grasper connection 20 (in the form of jaws with two opposing members as depicted in the Figures). Any other means known in the art that can grasp the mesh sheet 12 can be employed in the invention. The grasper connection 20 can contain more than two opposing members, as well as members of different lengths, widths, and shapes that will maximize the grasping function. In one aspect of the invention, once stapled to the abdominal wall, this grasper connection 20 can be released (if not previously released) and the contact mechanism can then be withdrawn back into the instrument 1 and removed from the port.

In one aspect of the invention, this grasping means can be supplemented by using a bonding agent that slowly degrades on contact with a body fluid. The boding agent is typically placed between the elements 18 and the mesh sheet 12. The amount of the bonding agent used (and the location placed) will depend on the expected time in which contact is needed to attach the mesh sheet. In one aspect of the invention, the boding agent can be used instead of the grasping means.

The medical instrument 1 of the invention also contains handling means. The handling means is that part of the device where the user holds and operates the instrument 1. Any handling means known in the art can be used in the invention, including the handle 4 depicted in the Figures.

The instrument 1 also contains optional means for steering. In this aspect of the invention, the instrument 1 can be made steerable to allow the instrument of the invention to be steered to the desired location in the body (i.e., the abdominal wall) using any suitable means known in the art. Suitable means include any mechanism that allows a user of the device to control the direction of the instrument. Examples of suitable steering means include those described in U.S. Pat. Nos. 5,857,996 and 5,399,164, the disclosures of which are incorporated herein by reference.

The instrument of the invention also contains triggering means. The triggering means is used to activate or trigger the holding means described above to expand it from the compressed position in inner housing 10. As well, the triggering means can be used to “pull” the contact mechanism into the inner housing 10 from its expanded position. Any suitable triggering means known in the art can be used in the invention, including the trigger 24 depicted in the Figures.

The medical instrument 1 of the invention can be used in the following manner. The user grasps the handling means and inserts the instrument 1 into the body (through a port previously made). The instrument 1 is gradually inserted until the end of the instrument is proximate the part of the abdominal wall (or other body part) that needs repairing. If necessary, the end of the instrument is steered until it is in the desired position.

The user then actuates the triggering means. This action causes the inner housing 10 to be advanced longitudinally relative to the outer housing 2. Advancing the inner housing 10 causes the contact mechanism to exit the open end of the inner housing 10, thereby extending the elements of the contact mechanism. As the trigger continues to be actuated, the contact mechanism continues to be pushes forward until it contacts the mesh sheet that has already been placed against the abdominal wall. If the mesh sheet has been pre-loaded in the instrument 1, such that the grasper means in the center of the instrument grasps the approximate center of the mesh, actuating the triggering means will open the contact mechanism and the attached mesh sheet 12 against the abdominal wall or other body surface. Further activation will open the grasper means and release the mesh.

The mesh sheet 12 is then positioned in the desired location. The elements of the contact mechanism serve to hold the mesh sheet 12 in place and the contact mechanism remains in contact with the mesh sheet until it is attached (i.e., stapled) to the abdominal wall. Where the instrument of the invention contains attaching means, such attaching means are then used to attach the mesh sheet in place.

Once the mesh sheet has been attached, the contact mechanism is withdrawn by actuating the triggering means again. This action causes the elements and the contact mechanism to be drawn back into the inner housing 10 and the inner housing then continues to be drawn back into the outer housing 2. At this point, the instrument can then be withdrawn through the incision/port.

In one aspect of the invention, the medical instrument is used during a process of attaching a mesh sheet to an abdominal wall during a hernia operation. In this procedure, the abdomen of the patient is insufflated. A first trochar can be then inserted and then used to introduce a light source and video camera to illuminate the hernia sac to locate the thinnest avascular entry point for creating the laparoscopic entry port(s).

The instrument of the invention is then inserted into the laparoscopic entry port in its closed position. The area of the hernia defect is then identified by viewing the above video image. The user then operates the instrument as indicated above to position the mesh sheet to the desired location of the hernia. A stapling device can then be introduced through an adjacent port to secure the mesh in place if the instrument of the invention does not contain the stapling device as an attachment means.

In one aspect of the invention, the elements can be configured to fold upon themselves. Here, the elements (which are made of a first portion and a second portion) initially extend from the open end of inner housing 10 in an extended position. When fully extended, the elements are compressed by folding the first portion back on the second portion. The mesh sheet is attached to the first portion and is held in place by first portion, thereby leaving the second portion of the elements not attached to the mesh sheet.

In another variation, the elements 18 are formed when the outer tube is compressed along its linear axis, such that a portion of the outer tube, into which 2 or more linear slits have been made, fold outwardly upon itself in an accordion type manner to create contact elements that are generally perpendicular to the long axis of the said outer tube. Above linear compression of the outer tube may be accomplished by attaching the distal end of the inner tube to the distal end of the outer tube, which is made of a flexible material, such as polyethylene. Holding the inner tube and pushing forward on the inner tube will cause the contact elements to deploy.

Using the instrument of the invention provides several advantages. The instrument quickly and easily places the mesh sheet against the body surface while the surgeon is attaching it. The instrument also keeps the mesh sheet substantially contiguous with the body surface during the attachment by reducing or preventing the sheeting from flapping around since the instrument holds it at more than one or two locations. The instrument is also an easily released from the mesh sheet once it has been attached. Finally, the instruments can be easily used with mesh sheets that have a backing or coating (such as silicone or Teflon) because of the instrument's ability to effectively grip the material with the grasping means.

In addition to any previously indicated variation, numerous other modifications and alternative arrangements may be devised by those skilled in the art without departing from the spirit and scope of the invention and appended claims are intended to cover such modifications and arrangements. Thus, while the invention has been described above with particularity and detail in connection with what is presently deemed to be the most practical and preferred aspects of the invention, it will be apparent to those of ordinary skill in the art that numerous modifications, including but not limited to, form, function, manner of operation and use may be made without departing from the principles and concepts set forth herein.

What is claimed is:

1. A medical device, comprising:
a housing;
a movable inner member contained within the housing; and
holding means contained within the movable inner member in a compressed configuration, said holding means capable of expanding outside the inner member to hold a flexible piece of material in at least three locations while the material is attached to an internal surface of the body.

2. The device of claim 1, wherein the holding means comprises an expandable and retractable contact mechanism.

3. The device of claim 2, wherein the contact mechanism contains an element for each location where the device holds the flexible piece of material.

4. The device of claim 3, wherein the number of elements ranges from 3 to 12.

5. The device of claim 1, wherein the holding means also holds the piece of material substantially contiguous with the internal surface.

6. The device of claim 1, wherein the holding means retains the piece of material in the inner member while in the compressed configuration.

7. The device of claim 1, further comprising means for grasping the flexible piece of material.

8. The device of claim 1, further comprising means for attaching the piece of material to the internal surface.

9. The device of claim 1, wherein the piece of material comprises a surgical mesh sheet.

10. A medical device, comprising:
a housing;
a movable inner member contained within the housing; and
holding means contained within the movable inner member in a compressed configuration, said holding means capable of expanding outside the inner member to hold a flexible piece of material substantially flat while the material is attached to an internal surface of the body.

11. The device of claim 10, wherein the contact mechanism contains an element for each location where the device holds the flexible piece of material.

12. The device of claim 10, wherein the number of elements ranges from 3 to 12.

13. The device of claim 10, wherein the holding means also holds the piece of material substantially contiguous with the internal surface.

15. The device of claim 10, further comprising means for grasping the flexible piece of material.

16. The device of claim 15, wherein the grasping means grasps the flexible piece of material near the center.

17. A medical device for positioning a flexible piece of material to an internal surface of the body, the device comprising means for holding the piece of material substantially contiguous to the internal surface.

18. A laparoscopic system containing a medical device, the medical device comprising means for holding the piece of material substantially contiguous to the internal surface.

19. A method for repairing a defect in an internal surface of the body, comprising:
placing a flexible piece of material against an internal surface of the body;
holding the material against that surface in at least three locations; and
attaching the flexible piece of material to that surface.

23. The method of claim 22, wherein the material is held in 3 to 12 locations.

24. The method of claim 23, wherein the flexible piece of material is held substantially contiguous to the internal surface.

24. The method of claim 22, wherein the flexible piece of material is attached without changing the locations at which it is held.

25. A method, comprising:
placing a flexible piece of material against an internal surface of the body;
using a medical device containing holding means expanding outside the device to hold the flexible piece of material in at least three locations; and
attaching the flexible piece of material to that surface.

26. The method of claim 25, wherein the material is held in 3 to 12 locations.

27. The method of claim 25, wherein the flexible piece of material is held substantially contiguous to the internal surface.

28. The method of claim 25, wherein the flexible piece of material is attached without changing the locations at which it is held.

29. The method of claim 25, wherein the holding means contains an element for each location where the device holds the piece of material.
